(19) United States
(12) Patent Application Publication
(10) Pub. No.: US 2012/0282678 A1
(43) Pub. Date: Nov. 8, 2012

(54) METHOD FOR RECOVERING INERT OR LIVING MICROPARTICLES AND USE AND INSTALLATION OF SAME

(75) Inventors: Jean-Yves Champagne, Lyon (FR); Francois Rene, Saussan (FR)

(73) Assignees: Institut Français de Recherche Pour L'Exploitation de la Mer Ifremer, Issy les Moulineaux (FR); INSTITUT NATIONAL DES SCIENCES APPLIQUEES, Villeurbanne (FR)

(21) Appl. No.: 13/508,872

(22) PCT Filed: Nov. 9, 2010
(86) PCT No.: PCT/FR2010/052401

§ 371 Date: Jul. 20, 2012

(30) Foreign Application Priority Data
Nov. 9, 2009 (FR) 09/57898

(51) Int. Cl. C02F 1/24; C12N 1/12; B01D 19/02; C02F 103/20; C02F 103/08

(52) U.S. Cl. 435/257.1; 210/704; 210/188

(57) ABSTRACT

A method for recovering inert or living microparticles in which a rising liquid column is set up under negative pressure of an aqueous effluent that includes inert or living microparticles, and a foam is separated at the top of the column into a multiphase effluent enriched with inert or living microparticles relative to the aqueous effluent and into a mostly liquid effluent depleted of inert or living microparticles relative to the aqueous effluent. The invention also concerns the different applications of this method and an installation implementing this method.

The present invention concerns a method and an installation for recovering inert or living microparticles, preferably photosynthetic micro-organisms.

In the present invention, the generic term photosynthetic micro-organisms will be used to designate microscopic photosynthetic organisms which may be single-celled or undifferentiated multi-celled. The present invention will be more particularly described on the basis of photosynthetic microorganisms such as microalgae, without this forming any kind of limitation. Also, for the recovery of photosynthetic microorganisms, the term <<harvesting method>> will be used.

In addition, by inert microparticles is meant colloids (dissolved organic matter), micro-flocculants, sludge or silica compounds.

There is currently a sharp rise in the culture of photosynthetic microorganisms on account of the exceptional potential of these simple organisms which are capable of capturing carbon gas and all other gaseous compounds considered to be pollutants i.e. NOₓ compounds (such as NO₂, NO₃, and other gaseous or atomized nitrogen-containing compounds) and SOₓ compounds (such as SO₂, S₂O₄ and other gaseous or atomized sulfur-containing compounds whether sulfurous or hydrogenated) to synthesize their organic matter by photosynthesis with extensive surface efficacy (at least ten times higher in terms of recoverable production compared with cultivated land plant productions). Photosynthetic microorganisms at the present time are therefore chiefly cultivated in open systems such as lagoons or ponds. More precisely, they can be cultivated in long glass tubes or in ponds, in salt or super-salted water or in fresh water, and thereby offer possible continuous harvesting. Recent technological developments also allow the culture of photosynthetic organisms in closed photo-bioreactors; this provides the advantage of implementing a fully controlled method for cultivating specific photosynthetic micro-organisms under aseptic conditions.

The interest in photosynthetic microorganisms has therefore undergone a particular increase in recent years owing to their use for the production of biofuels in particular, from oils extracted from microscopic algae present in plankton.

It is known that the production of biofuel based on sunflower seed, soybean or sugar cane generates high production costs. Photosynthetic micro-organisms on the other hand offer a yield that is thirty times higher than that of oilseeds (through their capacity to accumulate fatty acids which may represent up to 50% of their dry weight) with the additional advantage of not harming the environment.

Photosynthetic microorganisms therefore represent a non-competing alternative to food crops and additionally of great interest through:
their much higher potential for development;
the particularity of some species of photosynthetic microorganisms to produce lipid reserves of up to 70% of their weight when subjected to stresses such as nitrogen deprivation or a sudden increase in light intensity;
no need for zoo- and phytosanitary products for their culture.

Photosynthetic microorganisms have the additional advantage of containing recoverable co-products for various fields of application such as:
pharmaceuticals and agri-foods (vitamins, omega 3, antioxidants, sugars);
industry (silica, pigments); and
petrochemicals (lipids).

However one of the chief obstacles to the technical and economic development of the culture of photosynthetic microorganisms lies in their harvesting method which must be efficient and of low energy cost. Unlike land plants (generally multi-cell organisms of large size) which can easily be harvested using mechanical means and which do not generate too high energy costs, the harvesting of photosynthetic microorganisms comes up against the problem of the small size of the product to be harvested, namely a mean size of between 0.5 and 60 microns.

From the state of the art, the following methods for harvesting photosynthetic microorganisms are known:
centrifugation, an efficient but costly technique: the harvesting of one to two kilos of dry algae requires the centrifugation of one tonne of culture medium (optimal, maximum density of harvested algae cultures at the present time);
flocculation represents an efficient technique from an energy viewpoint, but it requires the implementing of prior operations, in particular:
   spontaneous flocculation: a lengthy operation and the results obtained are not very reliable since this operation may jeopardize the quality of the harvested products;
   bio-flocculation by mixing with a bacterial culture: a technique which proves to be costly in terms of energy and scarcely reliable;
   cold shock flocculation, efficient but very high in energy costs;
harvesting by tangential filtration, a costly technique in terms of energy which often destroys the prominent appendages of the cell walls of photosynthetic microorganisms; in addition the presence of algae with siliceous backbone may rapidly damage the filtering walls thereby limiting their lifetime.

These methods for harvesting photosynthetic microorganisms prove to be not only costly in terms of energy but may also be destructive of the harvested photosynthetic microorganisms. In addition, they require permanent maintenance of the filtering screens in particular if diatoms are present having siliceous capsules, the thecae of these algae destroying the filtering screens of mesh size between 1 and 30 microns.

The present invention overcomes these drawbacks by proposing a method for recovering inert or living microparticles that is fully adapted to the harvesting of photosynthetic microorganisms such as microalgae, since the method:
optimizes recovery energy costs, in particular for the harvesting of photosynthetic microorganisms by pre-concentration;
preserves the integrity of photosynthetic microorganisms;
requires only low management and implementation costs;
makes continuous harvesting possible by permanent trimming of the cultures.

The method for recovering inert or living microparticles according to the present invention comprises the following steps:
a) a rising liquid column is formed under negative pressure of an aqueous effluent comprising inert or living microparticles;
   a gas phase is injected into the bottom side of the column, the said gas phase being distributed within the column in the form of bubbles; and
   a negative pressure is set up on the top side of the column,
      so that the diameter of the bubbles increases as and when they migrate towards the top of the column and so as to obtain a foam on the top side of the column, the foam being formed of a multiphase mixture of the aqueous effluent and the gas phase;
b) the foam at the top of the column is separated into:
   a multiphase effluent enriched with inert or living microparticles relative to the aqueous effluent, the said multiphase effluent being mostly formed of the gases of the gas phase whose composition may have been modified through gas exchanges occurring in the column;
   a mostly liquid effluent depleted of inert or living microparticles relative to the aqueous effluent and which moves down the column;
c) the inert or living microparticles contained in the multiphase effluent are concentrated by separation of the phases of the said multiphase effluent into:
   a concentrate formed of a liquid comprising the inert or living microparticles;
   a gas phase formed of the gases of the gas phase whose composition may have been modified through gas exchanges occurring in the column;
d) the inert or living microparticles are recovered by means of a flocculation step, followed by a sedimentation step. In the present invention, the term <<effluent>> is used in its more general meaning to designate any fluid emanating from a multiphase source, namely formed of a mixture of liquid, gas and solid elements, the proportion of each these three states possibly being most variable.

At step b) of the method of the invention, by:
<<multiphase effluent mostly consisting of gases of the gas phase>> is meant an effluent whose proportion of gases present in this effluent is equal to or more than 90%;
<<mostly liquid effluent>> is meant an effluent whose proportion of liquid present in this effluent is equal to or more than 80%.

According to this method, inert or living microparticles present in concentrations of between 0.1 g/m³ and 10 000 g/m³ in an aqueous effluent can be recovered. The concentration of inert or living microparticles in the aqueous effluent will depend on the type of application for which the method is intended.

Indeed, the method of the invention has the advantage of being able to be implemented over a very broad range of concentrations of living or inert microparticles present in an aqueous effluent, and hence it can be used in most varied fields of technical application which are described below.

Also, since the method of the invention is able to function continuously, it is possible to adapt the recovery rate of the inert or living microparticles present in an aqueous effluent in relation to the desired energy cost; this depending on the field of application of the method. In particular, owing to possible closed loop functioning, the method of the invention can be implemented a certain number of times on the aqueous effluent containing inert or living microparticles so as to increase the recovery rate of the said inert or living microparticles.

The method of the invention allows the recovery of inert or living microparticles able to be concentrated between 10 and 30 times, even between 100 and 1000 times, relative to those present in the aqueous effluent to which the method is applied.

The method of the invention can be used in the field of application of mains water treatment by recovering flows of bacteria, colloids, residual inert microparticles such as clays, sludge or silica compounds, the concentration of microparticles in the aqueous effluent being between 5 and 10 g/L. The method allows pure water to be obtained after recovery of the said microparticles at a residual concentration of these microparticles of the order of 10⁻³ g/L.

The method of the invention can be used for off-shore oil drilling, namely for pre-filtering (80-90%) the seawater used to expel crude oil from oil-bearing parent rocks. For this use, the concentration of microparticles in the aqueous effluent is of the order of 0.1 g/m³. It may effectively be sought to purify open sea or shoreline water of any microparticle having a size greater than the pore size of the parent rock i.e. of the order of 5 μm. This use of the method allows low-cost pre-filtering of seawater, before total filtration using a more costly and more fragile process such as tangential filtration.

Another application of the method can concern the field of shellfish hatcheries, namely for separating the nutrient microalgae present in the rearing water of said hatcheries before ultra-violet sterilization (the medium to be sterilized having to be transparent). By means of this method, the algae thus separated remain living and can be re-injected into the rearing water circuit once sterilization has been carried out. For this application, the concentration of microparticles in the aqueous effluent may be between 10 g/m³ and 100 g/m³.

One preferred use of the method of the invention is the harvesting of microalgae, the concentration of microalgae in the aqueous effluent possibly reaching between 100 g/m³ and 10 000 g/m³, preferably between 5 000 g/m³ and 10 000 g/m³. With the method of the invention, it is possible to obtain concentrated microalgae at a concentration of between 10 and 20 g/L, and thereby to concentrate the initial aqueous effluent 20 to 30 times when the concentration of the biomass is of the order of 0.5 to 1 g of dry matter per litre of culture, even 100- to 1000-fold concentration when the concentration of microalgae in the aqueous effluent is lower, namely lower than 0.05 g/L.

Another application of the method of the invention consists of treating aquarium waters, or pond waters for rearing aquaculture organisms through the filtration of microparticles (colloids and fine particles such as proteins) having a size of the order of 0.01 μm, so as to obtain <<pure residual waters>> of the order of 0.001 g/L of matter in suspension in the aquarium water.

Therefore, one subject of the present invention is a method for treating pond water used for farming aquaculture organisms which comprises the following steps:
a) a rising liquid column is set up under negative pressure of an aqueous effluent from a first source, the said first source being an aquaculture organism rearing pond into which oxygen is optionally injected, the said aqueous effluent comprising faeces, colloids, fine particles such as proteins, and dissolved gases such as ammonia, nitrogen gas and carbon dioxide, produced by the aquaculture organisms,
   by injecting into the bottom side of the column a gas phase formed of air and optionally ozone, the said gas phase being distributed within the column in the form of bubbles, and
   by setting up negative pressure on the top side of the column,
so that the bubbles increase in diameter during their migration towards the top of the column, and so as to obtain a foam on the top side of the column, the foam consisting of a multiphase mixture of aqueous effluent and gas phase;
b) the foam at the top of column is separated into:
   a multiphase effluent enriched with faeces, colloids, fine particles, dissolved gases relative to the aqueous effluent, the said multiphase effluent being mostly formed of gases of the gas phase whose composition may have been modified through gas exchanges occurring in the column;
   a mostly liquid effluent moving down the column depleted of faeces, colloids and dissolved gases relative to the aqueous effluent and into which oxygen may optionally be injected;
c) the faeces, colloids, fine particles contained in the multiphase effluent are concentrated by separation of the phases of the said multiphase effluent into:
   a concentrate formed of a liquid comprising the faeces, colloids, fine particles,
   a gas phase consisting of the gases of the gas phase whose composition may have been modified through gas exchanges occurring in the column with the gases dissolved in the aqueous effluent,
d) the faeces, colloids and fine particles are recovered.

The injection of oxygen into the mostly liquid effluent moving down the column has the effect of renewing the oxygen present in the aquaculture organism rearing pond and is complementary to direct injection into the rearing pond which may also be made.

One preferred embodiment of the method for treating aquaculture organism rearing ponds is characterized in that the aqueous effluent also derives from a second source. This second source is an agitated bed bacterial filter in which the nitrogen-containing and optionally carbon-containing waste produced by the aquaculture organisms and derived from the first source is converted to a biofilm in nitrate particle form which is continuously released by the said agitated bed bacterial filter. According to this preferred embodiment of the method, after step d) of the method the faeces, colloids, fine particles and released biofilm are recovered.

The method of the invention is based in particular on the physical principle of adsorption, by surface tension, of the microparticles present in a liquid on the periphery of bubbles created by the injection of a gas phase e.g. air into the said liquid.

The method of the invention is therefore all the more efficient through the maintained negative pressure at the top of the column which allows the regular increase in the diameter of the bubbles created on the bottom side of the column as and when they migrate towards the top of the column, and hence the formation of a foam at the top of the column. This improves the intrinsic buoyancy of each bubble. It also allows the use at the bottom of the column of very small bubbles particularly absorbent of inert or living microparticles. These particles are therefore found in the foam obtained at the top of the column. The smaller the bubble size the greater the adsorption phenomenon between gas bubbles/inert or living microparticles in the aqueous effluent, and hence the better the recovery yield of inert or living microparticles when using the method of the invention. Therefore, in one preferred embodiment of the invention, a gas phase is injected generating bubbles as small as possible namely of size less than 5 mm, preferably less than 1 mm. Advantageously, a finely atomized gas phase is injected using a suitable device.

In addition, by means of this negative pressure maintained at the top of the column, gas phase bubbles of smaller diameter are able to rise to the top of the column which would be impossible under atmospheric pressure.

Also, it is to be noted that when the inert microparticles (e.g. microparticles of petroleum products) or living microparticles (e.g. microalgae containing oxygen) contain dissolved gases, the negative pressure activates the upward migration and extraction from the foam of a multiphase effluent enriched with inert or living microparticles through the expansion of these gases contained in the said microparticles.

In one embodiment of the invention, surfactants are added to the aqueous effluent, to improve the phenomenon of gas bubble/inert or living microparticle adsorption. They can be recovered by recycling through the foaming or skimming effect occurring at the top of the column.

The negative pressure can be set up using any depressurizing system. Advantageously, the pressure is between 0.3 10⁵ and 0.9 10⁵ Pa. In one preferred embodiment of the invention, the negative pressure is set up by means of a vacuum pump. The height of the column is preferably between 1 and 6 m. Therefore, in the field of application concerning the harvesting of microalgae, the contact pathway length and contact time of the aqueous effluent with the gas phase are well above those of conventional skimmers using floating baffles whose height does not exceed one metre (the total pathway length not exceeding two metres). The length is effectively at least equal to the height of the column and may reach 2 to 3 times this height (i.e. about 6 to 18 metres) owing to phenomena of bubble recirculation in the column. This pathway length and time are of prime importance for everything related to skimming and gas exchange effects.

The gas phase injected into the bottom side of the column may be air, carbon dioxide or any other suitable gas under overpressure or atmospheric pressure.

The separation of the phases of the multiphase effluent at step c) of the method of the invention can be conducted in a concentrating device designed so that:
the concentrate comprising the inert or living microparticles is evacuated under gravity towards a collecting pond of the said microparticles, optionally after passing through a settling chamber, and so that
the gas phase is aspirated.

In one preferred embodiment of the invention, the aspiration of the gas phase can be conducted using the same system which sets up the negative pressure at the top of the column, e.g. a vacuum pump. Most advantageously, the concentration device consists of a foam separator which comprises an anti-foam grid and a ballasted float system.

If the inert or living microparticles flocculate spontaneously, they can be recovered at step d) of the recovery method of the invention in the form of a flocculate using any adequate extraction system such as:
a pumping system e.g. peristaltic pump,
Archimedes screw.

In this respect, by spontaneous flocculation is meant spontaneous aggregation of microparticles into particles of larger size until spontaneous sedimentation. Microalgae flocculate spontaneously.

If the inert or living microparticles do not flocculate spontaneously, they can be collected using any concentration process known to persons skilled in the art, such as centrifugation.

It is to be noted that, most generally, the method of the invention described above may optionally be completed by at least one step chosen from among the steps of washing, centrifugation and drying.

The present invention also concerns an installation for recovering inert or living microparticles, preferably an installation for harvesting photosynthetic microorganisms for implementation of the method of the invention.

The installation for recovering living or inert microparticles according to the invention comprises:
a) a column comprising:
   i. two concentric tubes, the first tube being an outer tube and the second tube being an inner tube, arranged vertically thereby forming:
      an inner tubular enclosure in which, via a column inlet on the bottom side of the column, an aqueous effluent containing living or inert microparticles enters and circulates upwardly; and
      an outer tubular enclosure in which a mostly liquid effluent circulates downwardly that is depleted of living or inert microparticles relative to the aqueous effluent, and which leaves the column via a column outlet located on the bottom side of the column,
   ii. means for injecting a gas phase into the inner tubular enclosure, the outer tube being closed in its upper part above the open upper end of the inner tube thereby forming a space in which a foam is formed of a multiphase mixture of aqueous effluent and gas phase when implementing the method of the invention,
   iii. means for placing the column under negative pressure which ensures the aspiration of a multiphase effluent enriched with living or inert microparticles relative to the aqueous effluent and is mostly formed of gases of the gas phase whose composition may have been modified through gas exchanges occurring in the column,
b) a device for concentrating the living or inert microparticles contained in the multiphase effluent;
c) a device for recovering the said inert or living microparticles concentrated in the concentrating device.

Preferably, the installation for recovering living or inert microparticles according to the invention further comprises a level regulating and safeguarding device.

Advantageously, the concentrating device of the installation for recovering living or inert microparticles comprises a foam separator.

Preferably, the recovery device of the installation for recovering living or inert microparticles comprises a settling chamber, a collecting pond, a peristaltic pump and a recovery pond.

In one preferred embodiment of the invention, the installation for recovering living or inert microparticles comprises a plurality of columns, preferably about sixty, and one single means for lowering pressure. Most advantageously, the installation comprises a plurality of columns sized so that the flow rate of the aqueous effluent is 3 to 5 times the flow rate of the injected gas phase.

Preferably the installation of the invention for recovering living or inert microparticles also comprises as many level regulating and safeguarding devices as columns.

The installation for recovering living or inert microparticles according to the invention will be better understood with the help of the detailed description set forth below with reference to the appended drawing which, as a non-limiting example, illustrates one embodiment of said installation.

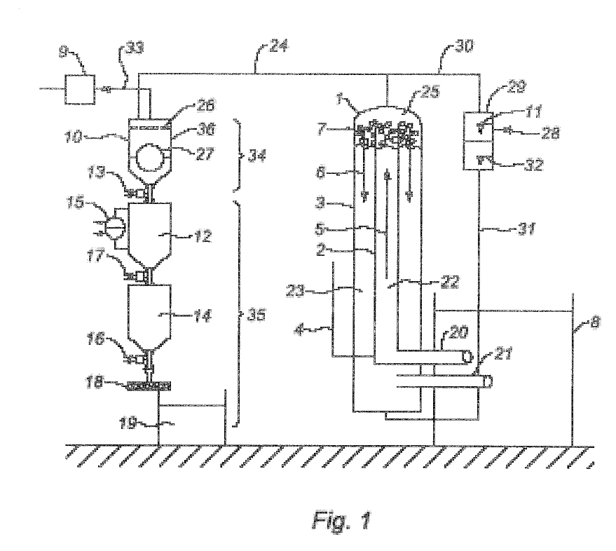

FIG. 1 schematically illustrates an installation for harvesting microalgae using the method of the invention. This installation comprises a column 1 formed of two concentric tubes 2, 3: a first outer tube 3 and a second inner tube 2. The two tubes 2, 3 are arranged vertically so as to provide an inner tubular enclosure 22 in which an aqueous effluent 5 comprising micro-algae circulates upwardly. The aqueous effluent enters via a column inlet 20 located on the bottom side of the column 1. This column inlet 20 is immersed in a microalgae culture pond 8. The column 1 is not immersed in the microalgae culture pond 8.

The two tubes 2, 3 also form an outer tubular enclosure 23 in which a mostly liquid effluent 6 circulates downwardly which is depleted of micro-algae relative to the aqueous effluent 5 and which leaves the column 1 via a column outlet 21 located on the bottom side of the said column 1. This column outlet 21 is immersed in the microalgae culture pond 8.

The column 1 comprises means 4 for injecting into the inner tubular enclosure 22 a gas phase formed of air. The outer tube 23 is closed in its upper part above the open upper end of the inner tube 22 so as to provide a space 25 in which a foam 7 is formed consisting of a multiphase mixture of aqueous effluent 5 and the gas phase when implementing the method of the invention.

In another embodiment of the invention, not illustrated, the aqueous effluent 5 is drawn off a storage installation e.g. a microalgae culture pond, by mechanical means and conveyed to the column inlet 20 of the column 1. The mostly liquid effluent 6 depleted of microalgae leaving via the column outlet 21 of the said column 1 can be re-introduced into this storage installation optionally using other mechanical means. In this case, the installation operates continuously. Therefore the microalgae contained in the mostly liquid effluent 6 depleted of microalgae are re-introduced into the storage installation and again drawn off from the storage installation and conveyed to the inlet 20 of the column 1.

In another embodiment of the invention, the mostly liquid effluent 6 leaving via the outlet 21 is sent to another storage unit.

In one embodiment of the invention, not illustrated, the column 1 and the column inlet 20 and column outlet 21 are immersed in a pond which contains inert or living microparticles to be recovered.

In the installation illustrated in FIG. 1, the depressurizing means 9 of the column 1 consist of a vacuum pump whose pressure is set at approximately 0.4 bar.

The installation illustrated in FIG. 1 comprises a concentrating device 34 which comprises a foam separator 10.

The multiphase effluent derived from the separation of foam 7 at the top of the column 1 is conveyed towards a foam separator 10 via a pipe 24. The foam separator 10 is in the form of a collecting tank 36 which comprises an anti-foam grid 26 and a ballasted float 27. The foam separator 10 is designed so that as and when the multiphase effluent is injected into the collecting tank 36, the separation of the phases at step c) of the method of the invention takes place by means of the vertical movement of the ballasted float 27. The foam separator 10 is connected to the vacuum pump 9 by a pipe 33. Therefore, at the foam separator 10, the gas phase that is formed of gases of the gas phase whose composition may have been modified through gas exchanges occurring in the column 1, is aspirated towards the vacuum pump 9 via the pipe 33. It is to be noted that the gas phase thus aspirated may pass through a bubbling chamber (not illustrated in FIG. 1) so as to protect the vacuum pump 9.

As illustrated in FIG. 1, the concentrating device 34 further comprises a motorized valve 13.

As illustrated in FIG. 1, the microalgae recovery device 35 comprises:
a settling chamber 12,
level sensors 15 of the settling chamber 12,
a motorized valve 17,
a collecting pond 14,
a motorized valve 16,
a peristaltic pump 18,
a recovery pond 19.

The installation for recovering microalgae illustrated in FIG. 1 also comprises a level regulating and safeguarding device 28. This level regulating and safeguarding device 28 consists of a cylinder-shaped chamber 29 of diameter about 7 cm and height of 10 cm, arranged facing the level of the set foam level 7 it is desired to obtain in the column 1.

The chamber 29 is connected:
in the upper part with the top part of the column 1 via first connection means 30, such as a flexible tube (of section between 0.5 and 3.5 cm for example),
in the lower part with the lower end of the column 1 opposite the top part of the column 1, via second connection means 31 such as a semi-rigid tube,
   so that the median level of the gas/liquid interface at the top of the column 1, by nature scarcely legible on account of the foam 7 and its agitation, is perfectly legible in this chamber 29 by means of an air/water interface free of foam 7 and agitation but accurately replicating the gas/liquid interface of the column 1 via the law of interconnected vessels (namely the fluid is at the same height in two interconnected vessels).
   The chamber 29 further comprises two level sensors 11,32:
a first sensor 11 to detect the upper limit of the level of the air/water interface which must not be exceeded in the chamber 29: upper set point;
a second sensor 32 to detect the lower limit of the level of the air/water interface which must not be exceeded in the chamber 29.

Most advantageously, the first sensor 11 is connected to a device controlling the flow rate of the multiphase effluent enriched with inert or living microparticles relative to the aqueous effluent (not illustrated in FIG. 1). Also advantageously, the second sensor 32 is connected to a device controlling the flow rate of the gas phase injected into the bottom of the column (not illustrated in FIG. 1). Preferably, the above-mentioned flow controlling devices are solenoid valves.

For example, if the level of the air/water interface in the chamber 29 is below the above-mentioned lower limit, this means that the multiphase effluent is not extracted at the top of the column 1, but on the contrary it is the mostly liquid effluent 6 which is extracted; this indicates ill-functioning of the installation of the invention. If the level of the air/water interface in the chamber 29 is below the above-mentioned lower limit, this means that the multiphase effluent is not extracted at the top of the column but it is air which is aspirated at the top of the column; this also indicates ill-functioning of the installation according to the invention.

Therefore, the regulating device 28 described above allows the monitoring of the proper functioning of the installation according to the invention.

In addition by means of this regulating device 28 described above, the flow rates of the multiphase effluent and of the gas phase injected into the bottom of the column can be regulated so that the multiphase effluent is indeed extracted at the top of the column by means of the depressurizing means 9 which set up the negative pressure throughout the installation of the invention, and it is not the mostly liquid effluent 6 depleted of inert or living microparticles which is extracted from the top of the column; this would risk damaging the depressurizing means 9 by flooding.

In one embodiment of the invention, not illustrated in FIG. 1, the installation comprises a plurality of columns 1, for example totalling sixty, whose column inlets 20 and column outlets 21 are immersed in the culture ponds 8. The columns 1 are not immersed in the culture ponds 8. In addition, a negative pressure is set up in the columns 1 by means of a single depressurizing means 9.

Therefore, according to this embodiment of the invention, the vacuum set up by the depressurizing means 9 is centralized, which ensures the resilience of the installation in the event of failure at one of the columns 1.

In addition, in one preferred embodiment, the installation for recovering living or inert microparticles according to the invention also comprises as many level regulating and safeguarding devices 28 as columns 1. Therefore in this embodiment, a separate level regulating and safeguarding device 28 is connected to each of the columns 1 such as described above.

A simulation was performed of the extraction costs obtained as a function of the volume of skimmed foam extracted on a microalgae recovery installation using the microalgae recovery method of the invention.

The extracted micro-algae species were the following:
Dunaliella Sp,
Tetraselmis Sp,
Nannocloropsis Sp,
Chlorococum Sp,
Skelotenema Sp,
Navicula Sp,
Spirulina platensis.

The results obtained are given in Table 1 below:

TABLE 1
Simulation of extraction costs

| | | | Micro- | | | Real |
|---|---|---|---|---|---|---|
| | | Micro- | algae | Energy | | volume |
| Extracted | | algae | dry | consump- | Extrac- | to be |
| foam | | concen- | matter | tion | tion | harvested |
| volume | | trate | weight | (KW/ | cost | per kg of |
| (L/30 min) | EC | (g/L) | (g) | 30 min) | ( [symbol] /Kg) | microalgae |
| 1 | 65.3 | 58.2 | 58.2 | 0.028 | 0.0392 | 17.19 |
| 3 | 37.7 | 33.6 | 100.7 | 0.028 | 0.0227 | 29.79 |
| 4 | 29.1 | 25.9 | 103.7 | 0.028 | 0.0220 | 38.57 |
| 10 | 14.8 | 13.2 | 131.9 | 0.028 | 0.0173 | 75.82 |
| 20 | 8.5 | 7.6 | 151.9 | 0.028 | 0.0150 | 131.71 |
| 35 | 5.8 | 5.2 | 180.8 | 0.028 | 0.0126 | 193.55 |
| 45 | 4.8 | 4.3 | 191.5 | 0.028 | 0.0119 | 235.04 |

In Table 1, the real volume to be harvested per kg of microalgae is the volume of microalgae concentrate obtained after step c) of the recovery method of the invention.

The term <<EC>> is the concentration factor whose definition is the following: concentration of the microalgae concentrate (expressed in g of dry matter/L of culture) divided by the concentration of the culture to be harvested (expressed in g of dry matter/L of culture).

The extraction cost is expressed in euros/kg.

According to the publication by Molina Grima E M et al. (2003) titled Recovery of microalgal biomass and metabolites: process options and economics, Biotechnology Advances 20:491-515, or the publication by Olaizola M. et al. (2003) Commercial development of microalgal biotechnology: from the test tube to the marketplace. Biomolecular Engineering 20: 459-466, the total production costs of microalgae are between 5 and 70 US dollars per kg of harvested dry matter for an extraction cost accounting for 25% to 30% of this total production cost. This represents an extraction cost of the order of 2 to 15 US dollars.

This indicates the very low extraction cost of microalgae by means of the recovery method and installation for recovering microalgae according to the present invention compared with the microalgae extraction costs which have been the subject of publications. The present invention is therefore particularly advantageous for the harvesting of microalgae.

Table 2 below gives a comparison of different parameters i.e. amortization, energy, labour and global extraction cost of microalgae using different techniques:
centrifugation,
flocculation,
bio-flocculation,
microalgae recovery device of the invention.

TABLE 2

| | | | | Device |
|---|---|---|---|---|
| [symbol] /Kg for dry | Centri- | Floccu- | Bio- | of the |
| matter at 100 g/L | fugation | lation | flocculation | invention |
| amortization over 5 yrs/ | 4 to 5 | 0.01 | 0.01 | 0.01 |
| weight (kg) of annually | | | | |
| harvested microalgae | | | | |
| energy | 1.5 | 0.01 | 0.01 | 0.0119 |
| labour | 1-1.5 | 2-5 | 2-8 | 0.05-0.5 |
| global extraction cost | 6-8 | 2.22-5.22 | 2.12-8.12 | 0.08-0.8 |

It follows from Table 2 that the microalgae recovery device is particularly advantageous from an economic viewpoint compared with other known microalgae recovery techniques that are used. By means of the recovery method and recovery device of the present invention, a significant reduction is ascertained in the extraction costs of microalgae.

The present invention therefore allows a crucial technological problem to be overcome as represented by the harvesting of microalgae from microalgae cultures. It proposes a method for recovering microalgae that is fully economical compared with known microalgae harvesting techniques. For the microalgae farming process, this provides a major advantage.

1. A method for recovering inert or living microparticles, comprising the following steps:
a rising liquid column under negative pressure is formed of an aqueous effluent comprising inert or living microparticles,
a gas phase is injected into the bottom side of the column, the said gas phase being distributed within the column in the form of bubbles; and
negative pressure is set up on the top side of the column,
so that the bubbles increase in diameter as they migrate towards the top of the column and so as to obtain, on the top side of the column, a foam consisting of a multiphase mixture of aqueous effluent and gas phase;
the foam at the top of the column is separated into:
a multiphase effluent enriched with inert or living microparticles relative to the aqueous effluent, the said multiphase effluent mostly consisting of gases of the gas phase whose composition may have been modified through gas exchanges occurring in the column, a mostly liquid effluent depleted of inert or living microparticles relative to the aqueous effluent and moving down the column,
the inert or living microparticles contained in the multiphase effluent are concentrated by separating the phases of the said multiphase effluent into:
a concentrate consisting of a liquid comprising the inert or living microparticles,
a gas phase consisting of gases of the gas phase whose composition may have been modified through gas exchanges occurring in the column,
d) the inert or living microparticles are recovered by means of a flocculation step followed by a sedimentation step.

2. The method for recovering inert or living microparticles according to claim 1, wherein the said inert or living microparticles are present in concentrations of between 0.1 g/m³ and 10000 g/m³.

3. The method for recovering inert or living microparticles according to claim 1, wherein a gas phase is injected generating bubbles of size less than 5 mm.

4. The method for recovering inert or living microparticles according to claim 1, wherein surfactants are added to the aqueous effluent.

5. The method for recovering inert or living microparticles according to claim 1, wherein the negative pressure is set up by means of a vacuum pump.

6. The method for recovering inert or living microparticles according to claim 1, wherein step c) is conducted by means of a foam separator which comprises an anti-foam grid and a ballasted float system.

7. The method for recovering inert or living microparticles according to claim 1, wherein said method is completed by at least one step chosen from among the steps of washing, centrifugation and drying.

8. A method for recovering flows of bacteria, colloids, inert residual microparticles, comprising treating mains water using the method of claim 1.

9. A method to pre-filter seawater used as fluid for expelling crude oil from oil-bearing parent rock, comprising exposing seawater used as fluid for expelling crude oil from oil-bearing parent rock to the method of claim 1.

10. A method for separating nutrient microalgae present in hatchery rearing waters, comprising exposing hatchery rearing waters to the method of claim 1.

11. A method for harvesting microalgae, comprising treating a source according to the method of claim 1.

12. A method for treating the water of aquariums or aquaculture organism rearing ponds, comprising exposing water of aquariums or aquaculture organism rearing ponds to the method of claim 1.

13. A method for treating the water of aquaculture organism rearing ponds which comprises the following steps:
forming a rising liquid column under negative pressure of an aqueous effluent derived from a first source, said first source being an aquaculture organism rearing pond into which oxygen is optionally injected, the said aqueous effluent comprising faeces, colloids, fine particles such as proteins, and dissolved gases such as ammonia, nitrogen gas and carbon dioxide produced by the aquaculture organisms,
infecting into the bottom side of the column a gas phase consisting of air and optionally ozone, the said gas phase being distributed within the column in the form of bubbles, and
setting up a negative pressure on the top side of the column,
so that the bubbles increase in diameter as they migrate towards the top of the column, and so as to obtain on the top side of the column a foam consisting of a multiphase mixture of aqueous effluent and the gas phase,
the foam at the top of the column is separated into:
a multiphase effluent enriched with faeces, colloids, fine particles, dissolved gases relative to the aqueous effluent, the said multiphase effluent mostly consisting of gases of the gas phase whose composition may have been modified through gas exchanges occurring in the column,
a mostly liquid effluent moving down the column which is depleted of faeces, colloids and dissolved gases compared with the aqueous effluent, and into which oxygen is optionally injected;
the faeces, colloids, fine particles contained in the multiphase effluent are concentrated by separation of the phases of the said multiphase effluent into:
a concentrate consisting of a liquid comprising faeces, colloids, the fine particles,
a gas phase which consists of the gases of the gas phase whose composition may have been modified through gas exchanges occurring in the column with the gases dissolved in the aqueous effluent,
the faeces, colloids, fine particles are recovered.

14. The method for treating the water of aquaculture organism rearing ponds according to claim 13, wherein the aqueous effluent also derives from a second source, the said second source being an agitated bed bacterial filter in which the nitrogen-containing and optionally carbon-containing waste produced by the aquaculture organisms in the first source is converted to a biofilm in particulate nitrate form which is continuously released by the said agitated bed bacterial filter, and in that after step d) the faeces, colloids, fine particles and released biofilm are recovered.

15. An installation for recovering living or inert microparticles comprising:
column comprising:
i. two concentric tubes, the first tube being an outer tube and the second tube being an inner tube, arranged vertically, thereby forming:
an inner tubular enclosure into which, via a column inlet located on the bottom side of the column, an aqueous effluent comprising living or inert microparticles enters and circulates upwardly, and
an outer tubular enclosure in which a mostly liquid effluent moves downwardly depleted of living or inert microparticles relative to the aqueous effluent and which leaves the column via a column outlet located on the bottom side of the column;
means for injecting a gas phase into the inner tubular enclosure,
the outer tube being closed in its upper part above the open upper end of the inner tube forming a space in which a foam is formed consisting of a multiphase mixture of the aqueous effluent and the gas phase when implementing the method according to claim 1;
means for depressurizing the said column and which ensure the aspiration of a multiphase effluent enriched with living or inert microparticles relative to the aqueous effluent and mostly consisting of gases of the gas phase whose composition may have been modified through gas exchanges occurring in the column,
a device for concentrating the living or inert microparticles contained in the multiphase effluent,
a device for recovering the said living or inert microparticles concentrated in the concentrating device.

16. The installation for recovering living or inert microparticles according to claim 15, wherein the installation further comprises a level regulating and safeguarding device.

17. The installation for recovering living or inert microparticles according to claim 15, wherein the concentrating device comprises a foam separator.

18. The installation for recovering living or inert microparticles according to claim 15, wherein the recovery device comprises a settling chamber, a collecting pond, a peristaltic pump and a recovery pond.

19. The installation for recovering living or inert microparticles according to claim 15, wherein the installation comprises a plurality of columns and one single depressurizing means.

20. The installation for recovering living or inert microparticles according to claim 19, wherein the said installation further comprises as many level regulating and safeguarding devices as columns.
